SEQUENCE LISTING (1 sequence)

SEQ ID NO: 1 - protein, 31 aa, synthetic construct
DGSVVVNKVSELPAGHGLNVNTLSYGDLAAD

(19) United States
(12) Patent Application Publication
(10) Pub. No.: US 2025/0154203 A1
(43) Pub. Date: May 15, 2025

(54) METHOD OF PRODUCING PEPTIDE DERIVED FROM CHAPERONIN 60.1

(71) Applicant: Revolo Biotherapeutics Limited, Great Shelford, Cambridgeshire (GB)

(72) Inventor: Pericles CALIAS, Melrose, MA (US)

(21) Appl. No.: 18/839,380

(22) PCT Filed: Feb. 16, 2023
(86) PCT No.: PCT/IB2023/000083

§ 371 Date: Aug. 16, 2024

Related U.S. Application Data
(60) Provisional application No. 63/311,354, filed on Feb. 17, 2022.

(51) Int. Cl. C07K 14/00; C07K 1/00; C07K 1/04; C07K 1/06

(52) U.S. Cl. CPC C07K 14/001; C07K 1/006; C07K 1/04; C07K 1/061

(57) ABSTRACT

An improved method of producing a peptide molecule as set forth in SEQ ID NO: 1 (DGSVVVNKVSELPAGHGLNVNTLSYGDLAAD) is described herein. According to some embodiments of the present disclosure, the method includes: forming a peptide by solid phase peptide synthesis (SPPS) on a resin; cleaving and deprotecting the peptide on the resin to form a crude product; purifying the crude product by column chromatography to collect eluant fractions; concentrating the eluant fractions to form a concentrated eluate; and isolating the peptide molecule from the concentrated eluate by precipitation and filtration.

### CROSS-REFERENCE TO RELATED APPLICATIONS

This application is national phase filing under 35 U.S.C. § 371 of International Application No. PCT/IB2023/000083 filed Feb. 16, 2023, which claims priority from U.S. Provisional Patent Application No. 63/311,354 filed Feb. 17, 2022, the entire contents of which are hereby incorporated by reference herein.

### FIELD OF THE INVENTION

The present disclosure in various aspects and embodiments relates to an improved method of synthesizing and purifying a peptide derived from Chaperonin 60.1.

### BACKGROUND OF THE INVENTION

Chaperonin polypeptides are a subgroup of heat shock polypeptides whose role in polypeptide folding is well known. There are two families of chaperonin polypeptide, the chaperonin 60 (approximately 60 kDa) and chaperonin 10 (approximately 10 kDa) families.¹ Conventionally, chaperonins assist polypeptide folding when the target polypeptide enters the central core of the ringed heptamers, and on the subsequent release of energy from ATP the target polypeptide is released from the central core by a conformational change in the chaperonin structure.²

More recently, some Chaperonin polypeptides have been shown to have a role in immune regulation. Mycobacterium tuberculosis (M. tuberculosis) produces Chaperonin 60.1 (Cpn60.1), a polypeptide that is named based on its amino acid sequence identity to other known chaperonins. International Patent Application, Publication Number WO2002/040037A2 disclosed pharmaceutical compositions comprising Cpn60.1 from M. tuberculosis (MtCpn60.1) and its encoding nucleic acid molecules. A variety of therapeutic uses for these molecules is also disclosed, including the treatment and/or prevention of autoimmune disorders, allergic conditions, conditions typified by a Th2-type immune response and conditions associated with eosinophilia. This application also disclosed a number of specific peptide fragments derivable from the whole length polypeptide which possess similar biological activity.

International Patent Application, Publication Number WO2009/106819A2 disclosed a series of novel peptides derivable from MtCpn60.1 including a peptide (designated as “Peptide 4”) having an amino acid sequence: DGSVVVNKVSELPAGHGLNVNTLSYGDLAAD (SEQ ID NO: 1). Peptide 4 exhibits anti-inflammatory activity and has been shown to significantly reduce the recruitment of eosinophils in an animal model of allergic airway inflammation.

Conventional methods as provided by the Almac Group,^{3,4} currently used to produce a peptide molecule as set forth in SEQ ID NO: 1 are inefficient and have low purity. Furthermore, the isolation with the convention methods requires lyophilization. A method that allows for precipitation of the peptide molecule would be preferable.

Therefore, there is a need for an improved method of synthesizing and purifying a peptide molecule as set forth in SEQ ID NO: 1.

### SUMMARY OF THE INVENTION

The present invention provides a process of synthesizing a peptide with SEQ ID NO: 1, comprising: (i) attaching an amino acid (AA) to a resin of a solid support via the AA's C terminus and wherein the N terminus of the amino acid is protected to avoid reaction at the N terminus to form a first solid support bound AA; (ii) deprotecting the N terminus of the first solid support bound AA by removing the protecting group; (iii) coupling a second AA with the first solid support bound AA wherein the C terminus of the second amino acid is coupled with the de-protected N terminus of the first solid support bound AA to form a second solid support bound AA, and wherein the second AA comprises a protected N terminus; (iv) repeating steps (ii) and (iii) to form the next solid support bound AA until a solid support bound AA sequence is formed; (v) cleaving the AA sequence from the solid support to yield a mixture of a peptide with a desired sequence ID; (vi) separating the peptide mixture from the solid support by filtration to yield a crude product; (vii) diluting the separated peptide mixture with solvents to form a precipitate, wherein the precipitate is isolated by filtration; (viii) subjecting the isolated precipitate to column chromatography to collect eluant fractions comprising a purified version of the peptide; (ix) concentrating the eluant fractions to form a concentrated eluate comprising the purified version of the peptide; and (x) isolating the peptide from the concentrated eluate by precipitation followed by filtration.

A preferred embodiment provides a process of synthesizing a peptide of SEQ ID NO: 1, wherein the SEQ ID NO: 1 is DGSVVVNKVSELPAGHGLNVNTLSYGDLAAD.

Another preferred embodiment provides a process wherein the C terminus of the AA Alanine (A) is attached to the resin bound amine of aspartic acid from the solid support by treating about 2.5 equivalent of the AA (A) with 2-(1H-benzotriazole-1-yl)-1,1,3,3-tetramethyluronium tetrafluoroborate (TBTU) and N,N-diisopropylethylamine (DIEA) in DMF, and further wherein the N-terminus of the AA (A) is protected by using Fmoc as the protecting group.

A further preferred embodiment provides a process wherein the Fmoc protected N-terminus of the dipeptide (AD) is deprotected by three consecutive treatments of the solid support bound dipeptide (AD) with a mixture of 10% piperidine in DMF and 0.15 M Oxyma.

Yet another preferred embodiment provides a process wherein A) the solid support bound dipeptide (AD) comprising a deprotected N-terminus is treated with about 2.5 equivalent of a second AA—Alanine (A) in the presence of N,N-diisopropylcarbodiamide (DIC) and ethyl 2-cyano-2-(hydroxyamino)acetate (Oxyma) in DMF, and further wherein the N-terminus of the second AA (A) is protected by using Fmoc as the protecting group. A further preferred embodiment provides a process wherein B) the N-terminus of the second AA (A) is deprotected by three consecutive treatments of the solid support bound AA's with a mixture of 10% piperidine in DMF and 0.15 M Oxyma, and wherein the solid support bound AA's with the deprotected N-terminus is sequentially treated with the steps in A) and B) until a solid supported peptide is formed with the SEQ ID NO:1.

Provided in a further preferred embodiment is a process wherein the peptide with SEQ ID NO:1 is obtained by cleaving the solid supported peptide with SEQ ID NO: 1 from the solid support by treating the solid supported peptide with SEQ ID NO:1 with an aqueous solution comprising trifluoroacetic acid (TFA) and triisopropylsilane (TIS), and separating the peptide with SEQ ID NO:1 from the solid support by passing the mixture through a filter wherein the peptide with SEQ ID NO:1 passes through the filter in to the filtrate, and wherein the filtered solid material is further washed up to eight times with the aqueous solution comprising TFA and TIS to yield a filtrate comprising the peptide with SEQ ID NO: 1. A further preferred embodiment provides a process wherein the elute is diluted with stepwise addition of methyl tert-butyl ether (MTBE), heptane, and MTBE, in a ratio of 1:0.75:1:1 by volume to yield the peptide with SEQ ID NO:1 as a precipitate.

Another embodiment provides a process wherein the SEQ ID NO:1 peptide precipitate is further subjected to a reverse phase high performance liquid column chromatography using a C₄ reversed phase column, wherein a pore size of the C₄ reversed phase column ranges from about 100-120 Å, and wherein a particle size of the C₄ reversed phase column is about 10 μm.

A preferred process of this embodiment provides a process wherein a mobile phase A is about 25 mM to about 50 mM ammonium acetate at a pH of about 7 to about 8.4, and wherein a mobile phase B is acetonitrile (ACN).

Yet another embodiment provides a process wherein purifying the peptide precipitate by a reverse phase high performance liquid column chromatography using a C₄ reversed phase column further comprises loading the C₄ reversed phase column to a concentration of about 23 mg of crude product per mL of stationary phase, and wherein the C₄ reverse phase column bed has a height of from about 20 cm to about 40 cm. Another preferred embodiment provides a process wherein the mobile phases are collected as eluants after passing through the C₄ reverse phase column, and further wherein the eluants are diluted with 10% of tris(hydroxymethyl)aminomethane (Tris) in water at a pH of about 7 to yield the peptide.

Another aspect of the present invention provides a process wherein isolating the purified product from the concentrated eluate includes diluting the concentrated eluate with 0.5× volume acetic acid (AcOH) premixed with ACN to form a reaction mixture. A preferred embodiment of this aspect provides a process wherein the peptide is diluted using MTBE to form a mixture.

Another preferred embodiment provides a process wherein isolating the peptide molecule from the mixture further comprises aging the reaction mixture for 30 minutes at 5° C. to yield a heterogenous mixture. Yet another preferred embodiment provides a process wherein the peptide from the heterogenous mixture is isolated by filtering the heterogenous mixture through a nylon membrane filter, and further wherein the nylon membrane filter is a 10 μm nylon membrane.

A further preferred embodiment provides a process wherein the isolated peptide is further washed with MTBE, the process further comprising humidifying the peptide to remove residual solvents. Yet another preferred embodiment provides a process wherein humidifying the peptide molecule includes humidifying the peptide molecule with wet N₂ until about 90% relative humidity is reached, followed by drying with a N₂ stream to yield the peptide in a dry form.

### BRIEF DESCRIPTION OF DRAWINGS

The accompanying drawings, which are incorporated in and constitute a part of this specification, exemplify various embodiments of the present invention and, together with the description, serve to explain and illustrate principles of the present disclosure. The drawings are intended only to illustrate major features of the exemplary embodiments in a diagrammatic manner.

FIG. 1 is an overlay of chromatograms showing purification of the peptide according to the improved method of the instant invention described herein (as depicted in the zoomed in chromatogram trace farther from the X axis, “Improved”, showing a major peak at approximately 18.4-18.6 minutes), compared to purification of the peptide according to a conventional method (as depicted in the chromatogram trace closer to the X axis and having significant peaks at 13.2, 17.6, 17.9, 18, 18.2, 18.5, and 19 minutes). Both chromatograms were acquired at 210 nm (+/−1 nm).

FIG. 2 is a normalized view of the chromatogram overlays of FIG. 1 with both chromatograms normalized on the main peak between about 18.4-18.6 on the X axis. Both chromatograms were acquired at 210 nm (+/−1 nm).

FIG. 3 is a mass spectrometry (MS) chromatogram of the material produced by conventional methods including the peptide (main peak) and labeled impurity peaks. The MS total ion chromatogram (TIC) chromatogram is for positive ions.

### DETAILED DESCRIPTION OF THE INVENTION

The present invention provides a process of synthesizing a peptide with SEQ ID NO: 1, comprising: (i) attaching an amino acid (AA) to a resin of a solid support via the AA's C terminus and wherein the N terminus of the amino acid is protected to avoid reaction at the N terminus to form a first solid support bound AA; (ii) deprotecting the N terminus of the first solid support bound AA by removing the protecting group; (iii) coupling a second AA with the first solid support bound AA wherein the C terminus of the second amino acid is coupled with the de-protected N terminus of the first solid support bound AA to form a second solid support bound AA, and wherein the second AA comprises a protected N terminus; (iv) repeating steps (ii) and (iii) to form the next solid support bound AA until a solid support bound AA sequence is formed; (v) cleaving the AA sequence from the solid support to yield a mixture of a peptide with a desired sequence ID; (vi) separating the peptide mixture from the solid support by filtration to yield a crude product; (vii) diluting the separated peptide mixture with solvents to form a precipitate, wherein the precipitate is isolated by filtration; (viii) subjecting the isolated precipitate to column chromatography to collect eluant fractions comprising a purified version of the peptide; (ix) concentrating the eluant fractions to form a concentrated eluate comprising the purified version of the peptide; and (x) isolating the peptide from the concentrated eluate by precipitation followed by filtration.

A preferred embodiment provides a process of synthesizing a peptide of SEQ ID NO: 1, wherein the SEQ ID NO:1 is DGSVVVNKVSELPAGHGLNVNTLSYGDLAAD.

Another preferred embodiment provides a process wherein the C terminus of the AA Alanine (A) is attached to the resin bound amine of aspartic acid from the solid support by treating about 2.5 equivalent of the AA (A) with 2-(1H-benzotriazole-1-yl)-1,1,3,3-tetramethyluronium tetrafluoroborate (TBTU) and N,N-diisopropylethylamine (DIEA) in DMF, and further wherein the N-terminus of the AA (A) is protected by using Fmoc as the protecting group.

A further preferred embodiment provides a process wherein the Fmoc protected N-terminus of the dipeptide (AD) is deprotected by three consecutive treatments of the solid support bound dipeptide (AD) with a mixture of 10% piperidine in DMF and 0.15 M Oxyma.

Yet another preferred embodiment provides a process wherein A) the solid support bound dipeptide (AD) comprising a deprotected N-terminus is treated with about 2.5 equivalent of a second AA—Alanine (A) in the presence of N,N-diisopropylcarbodiamide (DIC) and ethyl 2-cyano-2-(hydroxyamino)acetate (Oxyma) in DMF, and further wherein the N-terminus of the second AA (A) is protected by using Fmoc as the protecting group. A further preferred embodiment provides a process wherein B) the N-terminus of the second AA (A) is deprotected by three consecutive treatments of the solid support bound AA's with a mixture of 10% piperidine in DMF and 0.15 M Oxyma, and wherein the solid support bound AA's with the deprotected N-terminus is sequentially treated with the steps in A) and B) until a solid supported peptide is formed with the SEQ ID NO:1.

Provided in a further preferred embodiment is a process wherein the peptide with SEQ ID NO:1 is obtained by cleaving the solid supported peptide with SEQ ID NO:1 from the solid support by treating the solid supported peptide with SEQ ID NO:1 with an aqueous solution comprising trifluoroacetic acid (TFA) and triisopropylsilane (TIS), and separating the peptide with SEQ ID NO: 1 from the solid support by passing the mixture through a filter wherein the peptide with SEQ ID NO: 1 passes through the filter in to the filtrate, and wherein the filtered solid material is further washed up to eight times with the aqueous solution comprising TFA and TIS to yield a filtrate comprising the peptide with SEQ ID NO:1. A further preferred embodiment provides a process wherein the elute is diluted with stepwise addition of methyl tert-butyl ether (MTBE), heptane, and MTBE, in a ratio of 1:0.75:1:1 by volume to yield the peptide with SEQ ID NO:1 as a precipitate.

Another embodiment provides a process wherein the SEQ ID NO: 1 peptide precipitate is further subjected to a reverse phase high performance liquid column chromatography using a C₄ reversed phase column, wherein a pore size of the C₄ reversed phase column ranges from about 100-120 Å, and wherein a particle size of the C₄ reversed phase column is about 10 μm.

A preferred process of this embodiment provides a process wherein a mobile phase A is about 25 mM to about 50 mM ammonium acetate at a pH of about 7 to about 8.4, and wherein a mobile phase B is acetonitrile (ACN).

Yet another embodiment provides a process wherein purifying the peptide precipitate by a reverse phase high performance liquid column chromatography using a C₄ reversed phase column further comprises loading the C₄ reversed phase column to a concentration of about 23 mg of crude product per mL of stationary phase, and wherein the C₄ reverse phase column bed has a height of from about 20 cm to about 40 cm. Another preferred embodiment provides a process wherein the mobile phases are collected as eluants after passing through the C₄ reverse phase column, and further wherein the eluants are diluted with 10% of tris(hydroxymethyl)aminomethane (Tris) in water at a pH of about 7 to yield the peptide.

Another aspect of the present invention provides a process wherein isolating the purified product from the concentrated eluate includes diluting the concentrated eluate with 0.5× volume acetic acid (AcOH) premixed with ACN to form a reaction mixture. A preferred embodiment of this aspect provides a process wherein the peptide is diluted using MTBE to form a mixture.

Another preferred embodiment provides a process wherein isolating the peptide molecule from the mixture further comprises aging the reaction mixture for 30 minutes at 5° C. to yield a heterogenous mixture. Yet another preferred embodiment provides a process wherein the peptide from the heterogenous mixture is isolated by filtering the heterogenous mixture through a nylon membrane filter, and further wherein the nylon membrane filter is a 10 μm nylon membrane.

A further preferred embodiment provides a process wherein the isolated peptide is further washed with MTBE, the process further comprising humidifying the peptide to remove residual solvents. Yet another preferred embodiment provides a process wherein humidifying the peptide molecule includes humidifying the peptide molecule with wet N₂ until about 90% relative humidity is reached, followed by drying with a N₂ stream to yield the peptide in a dry form.

A method of producing a peptide molecule as set forth in SEQ ID NO:1 (DGSVVVNKVSELPAGHGLNVNTLSYGDLAAD) is described herein. The method generates the peptide molecule as set forth in SEQ ID NO:1 with improved purity compared to conventional methods for producing the peptide molecule as set forth in SEQ ID NO:1. In some embodiments, the method includes: forming a peptide by solid phase peptide synthesis on a resin; cleaving and deprotecting the peptide on the resin to form a crude product; purifying the crude product by column chromatography to collect eluant fractions; concentrating the eluant fractions to form a concentrated eluate; and isolating the peptide molecule from the concentrated eluate by precipitation and filtration.

The peptide according to SEQ ID NO:1 of the present disclosure is generated by solid phase peptide synthesis (SPPS). In SPPS, an amino acid or peptide group is bound to a solid support resin. Peptides are synthesized in the solid phase using chemistry by which amino acids are added from the C-terminus to the N-terminus. Thus, the amino acid or peptide group proximal to the C-terminus of a particular fragment is the first to be added to the resin. This occurs by reacting the C-terminus functionality of the amino acid or peptide group with complementary functionality on the resin support. The N-terminus side of the amino acid or peptide group is masked to prevent undesired side reactions. The amino acid or peptide group desirably also includes side chain protection as well. Then successive amino acids or peptide groups are attached to the support-bound peptide material until the peptide of interest is formed. Most of these also include side chain protection in accordance with conventional practices. With each successive coupling, the masking group at the N-terminus end of the resin bound peptide material is removed. This is then reacted with the C-terminus of the next amino acid whose N-terminus is masked. The product of solid phase synthesis is thus a peptide bound to a resin support. The support-bound peptide is then typically cleaved from the support and subject to further processing and/or purification.

Any type of support suitable in the practice of solid phase peptide synthesis can be used. In some embodiments, the support comprises a resin that can be made from one or more polymers, copolymers or combinations of polymers such as polyamide, polysulfamide, substituted polyethylenes, polyethyleneglycol, phenolic resins, polysaccharides, or polystyrene. The polymer support can also be any solid that is sufficiently insoluble and inert to solvents used in peptide synthesis. The solid support typically includes a linking moiety to which the growing peptide is coupled during synthesis and which can be cleaved under desired conditions to release the peptide from the support. Suitable solid supports can have linkers that are photo-cleavable, TFA-cleavable, HF-cleavable, fluoride ion-cleavable, reductively-cleavable; Pd(O)-cleavable; nucleophilically-cleavable; or radically-cleavable. Preferred linking moieties are cleavable under conditions such that the side-chain groups of the cleaved peptide are still substantially globally protected.

In some embodiments, fluorenylmethoxycarbonyl (Fmoc) based solid phase peptide synthesis (SPPS) is performed wherein the Fmoc group is used for temporary protection of the α-amino group. The Fmoc protecting group can be selectively cleaved from a peptide relative to the side chain protecting groups so that the side chain protection is left in place when the Fmoc is cleaved. This kind of selectivity is important during amino acid coupling to minimize side chain reactions. Additionally, the side chain protecting groups can be selectively cleaved to remove them relative to the Fmoc, leaving the Fmoc in place.

In some embodiments, coupling of the Fmoc-AA is carried out using about 2.5 equivalents of amino acid with N,N-diisopropylcarbodiimide (DIC) and ethyl 2-cyano-2-(hydroxyimino) acetate (Oxyma) in dimethylformamide (DMF).

In some embodiments, coupling Fmoc-AA comprises about 28 single couplings. In some embodiments, each couplings uses about 2.5 eq Fmoc-AA. In some embodiments, 12 of the 28 single couplings uses a coupling time of about 3 hours. In some embodiments, 12 of the 28 single couplings are carried out using about 2.5 equivalents of amino acid with DIC and Oxyma in DMF for 3 hours. In some embodiments, 16 of the 28 single couplings uses a coupling time of about 6 hours. In some embodiments, 16 of the 28 single couplings are carried out using about 2.5 equivalents of amino acid with DIC and Oxyma in DMF for 6 hours.

In some embodiments, about 70 eq of amino acid is needed to carry out coupling (28 single couplings, each coupling using about 2.5 eq of amino acid). On the other hand, conventional methods for purifying a peptide molecule as set forth in SEQ ID NO: 1 require 27 couplings using 4 equivalents of amino acid (18 single couplings for 45 minutes and 9 double couplings for 45 minutes) and therefore 144 equivalents are needed to carry out coupling. Relative to conventional methods for forming a peptide molecule as set forth in SEQ ID NO:1, the method described herein advantageously uses about 50% less raw materials than conventional methods, e.g., 144 eq Fmoc-AA in the conventional methods compared to 70 eq Fmoc-AA in the improved method.

After the coupling is determined to be complete, the coupling reaction mixture is washed with a solvent, and the coupling cycle is repeated for each of the subsequent amino acid residues of the peptide material. In order to couple the next amino acid, removal of the N-terminal protecting group (for example, an Fmoc group) from the resin-bound material is typically accomplished by treatment with a reagent that includes 20-50% (on a weight basis) piperidine in a solvent, such as N-methylpyrrolidone (NMP) or dimethylformamide (DMF). For example, in conventional methods for forming a peptide molecule as set forth in SEQ ID NO:1, Fmoc deprotecting is carried out using 20% piperidine in DMF and performed 2× with 10 volumes for 20 minutes each.

In some embodiments, Fmoc deprotecting is advantageously carried out using about 50% less piperidine relative to conventional methods. In some embodiments, Fmoc deprotecting is carried out using a 10% piperidine in DMF with 0.15 M Oxyma. In some embodiments, Fmoc deprotecting is performed 3× using 6.5 volumes for 10 minutes each.

After removal of the Fmoc protecting group, washing is performed to remove residual piperidine and Fmoc by-products (such as dibenzofulvene and its piperidine adduct). In some embodiments, washing the resin is carried out with a solvent. In some embodiments, the solvent is DMF. In some embodiments, washing with the solvent (e.g., DMF) is performed 9× using 6.5 volumes for 5 minutes each. In some embodiments, washing uses 59 volumes of solvent (e.g., DMF) per cycle. In some embodiments, the washing uses 1652 volumes of solvent in total (59 volumes of solvent for 28 cycles).

Conventional methods for forming a peptide molecule as set forth in SEQ ID NO: 1 require washing 20× with 5 volumes and therefore 100 volumes per cycle. In total, conventional methods use 700 volumes of solvent. Advantageously, the improved method described herein about 40% less volumes of solvent (e.g., DMF) is needed for the washing step than conventional methods.

In addition, conventional methods using solid phase peptide synthesis to form a peptide typically require a capping step with a capping reagent for a period of time (e.g., 10 minutes). In some embodiments, forming a peptide by solid phase peptide synthesis on a resin advantageously does not require a capping step after the coupling of the Fmoc-AA to block the ends of unreacted amino acids from reacting, thereby eliminating a step.

In some embodiments, after finalization of the synthesis, the next step is global deprotection, i.e., cleaving and deprotecting the peptide on the resin to form a crude product. In some embodiments, cleaving and deprotecting the peptide on the resin includes treating the resin with a solution comprising trifluoroacetic acid (TFA), H₂O, and triisopropylsilane (TIS). In some embodiments, treating the resin with the solution includes a 2.5 hour treatment with 7 volumes of the solution comprising trifluoroacetic acid (TFA), H₂O, and triisopropylsilane (TIS).

In some embodiments, the resin can then be removed by filtration and rinsed twice with TFA, thereby providing a filtrate with the peptide. In some embodiments, rinsing the resin twice is performed with 0.5 volumes of TFA. In some embodiments, cleaving the peptide from the resin only requires 8 volumes of solution (cleaving with 7 volumes of the cleavage solution and rinsing twice 0.5 volumes of TFA).

Conventional methods for forming a peptide molecule as set forth in SEQ ID NO: 1 typically require about 23 volumes to cleave the peptide (e.g., cleaving with 15.6 volumes of a solution including ethane-1,2-dithiol (EDT) and rinsing 3× with 2.5 volumes of TFA). Thus, cleaving the peptide as described herein provides for about a 65% volume reduction of cleavage solution and washing solution.

Furthermore, in some embodiments, the cleavage solution advantageously does not include ethane-1,2-dithiol (EDT). Conventional methods for forming a peptide molecule as set forth in SEQ ID NO: 1 typically require EDT in its cleavage solution, which is problematic as it is a pungent scavenger. Without being bound to any particular theory, EDT was found to be unnecessary for the cleavage solution in the method described herein, eliminating the pungent scavenger from the cleavage cocktail.

Following cleaving and deprotecting the peptide on the resin, which affords the peptide in solution, the peptide can be precipitated to form a crude product. In some embodiments, precipitating the crude product comprises stepwise adding methyl tert-butyl ether (MTBE) (6 volumes), heptane (8 volumes), and MTBE (8 volumes). In some embodiments, precipitating the crude product comprises stepwise adding 6 volumes of MTBE, 8 volumes of heptane, and 8 volumes of MTBE. Without being bound to any particular theory, stepwise addition of the antisolvents enhances filterability, whereas combining the antisolvents causes gelling of particles and slows down filtration.

In some embodiments, precipitating the crude product further comprises collecting a precipitate by filtration, washing the precipitate, and then drying to form the crude product. In some embodiments, washing the precipitate is performed 4× with 2 volumes of a solution of MTBE and heptane.

In some embodiments, precipitation of the crude product requires 30 volumes of antisolvent (stepwise addition of 6 volumes of MTBE, 8 volumes of heptane, 8 volumes of MTBE, and washing 4× with 2 volumes of a solution of MTBE and heptane after filtration). Conventional methods for purifying a peptide molecule as set forth in SEQ ID NO: 1 typically require about 59 volumes of antisolvent (e.g., precipitating with 45.3 volumes of MTBE and washing 3× with 4.8 volumes of MTBE). Thus, the precipitation as described herein advantageously affords an antisolvent volume reduction of about 50% compared to conventional methods.

Conventional methods for forming a peptide molecule as set forth in SEQ ID NO: 1 typically further require triturating the resulting solid from precipitation with another antisolvent (e.g., MTBE). For example, conventional triturating of the resulting solid from precipitation can be performed with 15 volumes of MTBE for 1.5 hours, isolated by filtration, washed 3× with 5 volumes of MTBE after filtration, and then re-dried. Advantageously, trituration of the precipitate is not needed in the improved method for purifying a peptide molecule as set forth in SEQ ID NO: 1 described herein. As a result, in some embodiments, the total volume of reactive moieties during global deprotection is 38 volumes (8 volumes during cleavage and 30 volumes during precipitation), which is about a 66% reduction in reactive moieties needed in conventional methods (23 volumes for cleavage, 59 volumes for precipitation, and 30 volumes for trituration).

In some embodiments, the next step following cleavage and deprotection of the resin-bound peptide is the purification of the crude peptide. In some embodiments, the crude peptide is purified by column chromatography to collect eluant fractions. In some embodiments, purifying the crude product by column chromatography comprises employing a reverse phase high performance liquid chromatography and a C₄ reversed phase column.

In some embodiments, the crude peptide is purified by HPLC employing YMC-Pack C4 (butyl) column or Kromasil C4 column. In some embodiments, a pore size of the C₄ reversed phase column is 100 Å, 120 Å, 200 Å, or 300 Å. Preferably, the pore size of the C₄ reversed phase column is 120 Å. In some embodiments, a particle size of the C₄ reversed phase column is 3 μm, 5 μm, or 10 μm. Preferably, the particle size of the C₄ reversed phase column is 10 μm.

In some embodiments, purifying the crude product by column chromatography further includes loading the C₄ reversed phase column to a concentration in the range of about 20 mg to about 35 mg of crude product per mL stationary phase, or about 23 mg to about 30 mg of crude product per mL stationary phase, or about 25 mg of crude product per mL stationary phase. In some embodiments, a column bed height is between about 10 cm to about 30 cm, about 15 cm to about 25 cm, about 20 cm to about 30 cm, or about 25 cm. In some embodiments, a column bed height is 25 cm.

In contrast, conventional methods for purifying a peptide molecule as set forth in SEQ ID NO:1 typically only allow for loading up to 3.2 mg of crude product per mL stationary phase, which is significantly less loading than the improved method described herein. For example, a front-eluting shoulder peak can result with the conventional methods, severely limiting loading. The higher loading afforded in the improved purification step enhances processability relative to the purification step in conventional methods for purifying a peptide molecule as set forth in SEQ ID NO:1.

In some embodiments, purifying the crude product by column chromatography includes at least two purification passes to collect the eluant fractions. In some embodiments, a mobile phase A is about 25-50 mM ammonium acetate at a pH of about 7-8.4. In some embodiments, a mobile phase B is acetonitrile (ACN). In some embodiments, the purification process involves two purification passes through chromatographic media, wherein a first chromatographic pass is carried out in an ammonium acetate gradient to provide a pH of about 7-8.4, and a second chromatographic pass is then carried out in a ACN gradient.

In some embodiments, purifying the crude product by column chromatography includes adding to the eluant fractions a stabilizing agent, like 10V % of tris(hydroxymethyl)aminomethane (Tris) at pH 7. Conventional methods for purifying a peptide molecule as set forth in SEQ ID NO:1 do not require adding a stabilizing agent to the eluant fractions. Instead, purification in the conventional methods result in gelled fractions which need to be re-dissolved by pH adjustment after visual inspection. Without being bound to any particular theory, prevention of gelling, as afforded by the purification step of the improved method, advantageously would be less invasive than the breakup of the gelled fractions required in the conventional methods for purifying a peptide molecule as set forth in SEQ ID NO:1.

In some embodiments, purifying the crude product by column chromatography further includes pooling the eluant fractions containing a product concentration and purity higher than a desired threshold to form a combined eluant fraction. In some embodiments, the crude peptide is purified to >90% (e.g., >91%, >92%, >93%, >94%, >95%, >96%, >97%, >98%, or >99%) and suitable fractions are pooled. In some embodiments, purity of peptides can be verified by reverse phase HPLC, followed by characterization of purified product (i.e., identity of peptides can be verified) by liquid chromatography/mass spectrometry (LC/MS) and/or Matrix-Assisted Laser Desorption Ionization (MALDI) mass spectrometry. Conventional methods for synthesizing and purifying a peptide molecule as set forth in SEQ ID NO:1 cannot generate a product with as high of a purity as the improved method described herein as evidenced in the Examples below.

In some embodiments, the next step following purifying the crude product by column chromatography to collect eluant fractions is concentrating the eluant fractions (or pooled eluant fractions) to form a concentrated eluate. In some embodiments, concentrating the eluant fractions or combined eluant fractions comprises loading the eluant fractions or combined eluant fractions onto a chromatographic column containing a polystyrene divinyl benzene resin or a C₄ material, and eluting with a solution of ACN containing ammonium acetate to form a concentrated eluate. Performing a concentration step can result in an increase of concentration of the desired peptide in the solvent by at least a factor of 2, 3, 4, 5, 6, 7, 8, 9, 10, 20, 30, 40, 50, 100, 200, 300, 400, or 500.

In some embodiments, an isolation step follows the concentration step. In some embodiments, isolating the peptide molecule from the concentrated eluate is carried out by precipitation and filtration. In some embodiments, isolating the purified product from the concentrated eluate includes diluting the concentrated eluate with 0.5× volume acetic acid (AcOH) premixed with ACN to form a reaction mixture. In some embodiments, isolating the peptide molecule from the concentrated eluate includes further diluting the reaction mixture with MTBE. In some embodiments, isolating the peptide molecule from the concentrated eluate includes aging the reaction mixture for 30 minutes or holding at 5° C.

In some embodiments, isolating the peptide molecule from the concentrated eluate includes, after aging, filtering the reaction mixture through a nylon membrane filter to isolate a precipitate. In some embodiments, the nylon membrane filter is a 10 μm nylon membrane filter. In some embodiments, isolating the peptide molecule from the concentrated eluate includes washing the precipitate with MTBE after filtering the reaction mixture through the nylon membrane filter. In some embodiments, after washing, the precipitate is dried.

In some embodiments, the method further includes, after precipitation, humidifying the peptide molecule to remove residual solvents. In some embodiments, humidifying the peptide molecule includes humidifying the peptide molecule with wet N₂ until about 90% relative humidity is reached, and drying with a N₂ stream.

Conventional methods of forming a peptide molecule as set forth in SEQ ID NO:1 do not use a concentration step. Instead, conventional methods pool and lyophilize fractions containing the desired peptide. Furthermore, conventional methods of forming a peptide molecule as set forth in SEQ ID NO:1 do not use a humidification step since the peptide is isolated via lyophilization.

### Synthetic Details

### Peptide Synthesis—General Procedure

The peptides according to the present invention are synthesized in multiple steps. The synthesis utilizes a solid-phase peptide synthesis (SPPS) wherein the first amino acid (AA) is covalently bound on a solid support material and synthesized step-by-step in a single reaction vessel utilizing selective protecting group chemistry. The first step involves binding an amino-protected amino acid to a solid phase material or resin (most commonly, low cross-linked polystyrene beads), forming a covalent bond between the carbonyl group of the amino acid (AA) and the resin. The covalent bond is an amido to an ester bond. The amino group which is protected by a protecting group like 9-fluorenylmethyloxycarbonyl group (Fmoc) and t-butyloxycarbonyl (Boc), is deprotected by treating the solid support bound AA with a mixture of 10% piperidin in DMF and 0.15 M Oxyma. The solid support bound AA now with the deprotected amino group is reacted/coupled with the carbonyl group of the next, N-protected, amino acid. This coupling yields a solid phase with a dipeptide. This foregoing cycle is repeated to form the desired peptide chain. After all reactions are complete, the synthesized peptide is cleaved from the solid support.

### Example 1

In this example, a peptide with SEQ ID No. 1 is being synthesized. As discussed in the general procedure, the SEQ ID No. 1 peptide is synthesized in multiple steps.

Step 1: The first step involves a solid-phase peptide synthesis (SPPS) wherein the carbonyl group/C terminus of amino acid (AA) 30—alanine (A) with its N terminus protected by a Fmoc group, is covalently bound to H-L-Asp (OtBu)-2CT-Resin which is the solid support with the AA 31 D already attached. This step involved treating the H-L-Asp (OtBu)-2CT-Resin (solid support) and AA (A) with 2-(1H-benzotriazole-1-yl)-1,1,3,3-tetramethyluronium tetrafluoroborate (TBTU) and N,N-diisopropylethylamine (DIEA) for about 2 hours at 23° C. temperature to yield Fmoc-AD-2CT-resin. The covalent bonding of the first AA was not monitored directly. The build-up of the correct sequence was checked periodically by running micro-cleavages and analyzing them by HPLC-MS.

Experimental procedure: Fmoc-L-Ala-OH (2.5 eq) and TBTU (2.44 eq) were weighed in an appropriate vial. Prior to coupling of the amino acid, DMF (c=0.25 mol/L for the AA) was added to the vial. After a clear solution was formed, DIEA (4.90 eq) was added and the activated AA was agitated for 2-10 min. The pre-activated AA was then transferred to the resin and agitated at RT for 120 min. The resin was washed 4 times with DMF [6.5 ml/g (resin), 5 min each].

Step 2: This step involved deprotection of the N-terminus nitrogen by removing the Fmoc protecting group. This deprotection step involves treating the solid support bound dipeptide (AD) with a with a mixture of 10% piperidin in DMF and 0.15 M Oxyma to yield a solid support bound dipeptide (AD) with a deprotected N-terminus. No direct monitoring of this step was performed. Analyses of micro-cleavages by HPLC-MS did not show any deletions of the next amino acid which would be a direct impurity generated by incomplete deFmoc of this step. This analysis was done in the R&D phase and was then neglected after confirming no impurities generated by this step.

Experimental procedure: The resin was treated three times with 10% piperidine in DMF containing 0.15 M Oxyma [6.5 ml/g (resin), 10 min each]. The resin was washed 5 times with DMF [6.5 ml/g (resin), 5 min each].

Step 3: This step involved treating the solid support bound dipeptide (AD) comprising a deprotected N-terminus with 2.5 equivalents of amino acid Alanine AA (A) wherein its N-terminus is protected with a Fmoc group along with N,N-diisopropylcarbodiamide (DIC) and ethyl 2-cyano-2-(hydroxyamino)acetate (Oxyma) in DMF for about 3 hours at a temperature ranging from 20 to 24° C. The covalent bonding of this AA was not monitored directly. The build-up of the correct sequence was checked periodically by running micro-cleavages and analyzing them by HPLC-MS.

Experimental procedure: Fmoc-Ala-OH (2.5 eq) and OxymaPure (2.5 eq) were weighed in an appropriate vial and dissolved with DMF (c=0.25 mol/L). Prior to coupling of the amino acid, DIC (3.75 eq) was added to the vial and the solution was agitated for 2-15 min. The activated AA was transferred to the resin and shaken at RT for 180 min. The resin was washed 4 times with DMF [6.5 ml/g (resin), 5 min each. The Example conditions used are provided in Tables 1-7 below. The SPPS Sequence is shown in Table 1 below. Example protocols for SPPS are provided in Table 2, and global deprotection protocols are in Table 3. The 1^{st} and 2^{nd} pass purification conditions are in Table 4 and Table 5, respectively. The concentration pass is in Table 6, and Table 7 provide precipitation, filtration, and humidification protocols.

TABLE 1
SPPS Sequence.

| Cycle | AA | Coupling conditions |
|---|---|---|
| 0 | H-L-Asp(OtBu)-2CT-Resin | Swelling |
| 1 | Fmoc-L-Ala * H2O | TBTU/DIEA, 2 h |
| 2 | Fmoc-L-Ala * H2O | DIC/Oxyma, 3 h |
| 3 | Fmoc-L-Leu-OH | DIC/Oxyma, 3 h |
| 4 | Fmoc-L-Asp(OtBu)-OH | DIC/Oxyma, 3 h |
| 5 | Fmoc-Gly-OH | DIC/Oxyma, 3 h |
| 6 | Fmoc-L-Tyr(tBu)-OH | DIC/Oxyma, 3 h |
| 7 | Fmoc-L-Ser(tBu)-OH | DIC/Oxyma, 3 h |
| 8 | Fmoc-L-Leu-OH | DIC/Oxyma, 3 h |
| 9 | Fmoc-L-Asn(Trt)-L-Thr(psiMe, Mepro)-OH | DIC/Oxyma, 6 h |
| 10 | Fmoc-L-Val-OH | DIC/Oxyma, 6 h |
| 11 | Fmoc-L-Asn(Trt)-OH | DIC/Oxyma, 6 h |
| 12 | Fmoc-L-Leu-OH | DIC/Oxyma, 6 h |
| 13 | Fmoc-Gly-OH | DIC/Oxyma, 6 h |
| 14 | Fmoc-L-His(Trt)-OH | DIC/HOPO, 3 h |
| 15 | Fmoc-Gly-OH | DIC/Oxyma, 6 h |
| 16 | Fmoc-L-Ala * H2O | DIC/Oxyma, 6 h |
| 17 | Fmoc-L-Pro-OH * H2O | DIC/Oxyma, 6 h |
| 18 | Fmoc-L-Leu-OH | DIC/Oxyma, 6 h |
| 19 | Fmoc-L-Glu(OtBu)-OH * H2O | DIC/Oxyma, 6 h |
| 20 | Fmoc-L-Val-L-Ser(psiMe, Mepro)-OH | DIC/Oxyma, 6 h |
| 21 | Fmoc-L-Lys(Boc)-OH | DIC/Oxyma, 3 h |
| 22 | Fmoc-L-Asn(Trt)-OH | DIC/Oxyma, 6 h |
| 23 | Fmoc-L-Val-OH | DIC/Oxyma, 6 h |
| 24 | Fmoc-L-Val-OH | DIC/Oxyma, 6 h |
| 25 | Fmoc-L-Val-OH | DIC/Oxyma, 3 h |
| 26 | Fmoc-L-Ser(tBu)-OH | DIC/Oxyma, 6 h |
| 27 | Fmoc-Gly-OH | DIC/Oxyma, 6 h |
| 28 | Boc-L-Asp(OtBu)-OH | DIC/Oxyma, 3 h, |
| | | noDeFmoc |
| 29 | — | Final Wash |

TABLE 2
Protocols for SPPS.
Swelling

| | |
|---|---|
| Resin-Swell | 4 × 5 min, 6.5 mL/g (inital resin) DMF |

DeFmoc

| | |
|---|---|
| Deprotection | 10% Piperidine in DMF + 0.15M Oxyma, |
| | 6.5 mL/g (initial resin), 3 × 10 min |
| Wash (Batch) | 5 × 5 min, 6.5 mL/g(resin) DMF |

Coupling
TBTU/DIEA, 2 h (first AA)

| | |
|---|---|
| Coupling | 2.50 eq AA, 2.44 eq TBTU, 4.90 eq DIEA, |
| | 0.25 mol/L (on AA) in DMF |
| | 2-10 min preactivation |
| | 2 h Coupling time |
| Wash | 4 × 5 min, 6.5 mL/g (initial resin) DMF |

DIC/Oxyma, 3 h

| | |
|---|---|
| Coupling | 3.75 eq DIC, 2.50 eq AA, 2.5 eq OxymaPure, |
| | 0.25 mol/L (on AA) in DMF |
| | 2-10 min preactivation |
| | 3 h Coupling time |
| Wash | 4 × 5 min, 6.5 mL/g (initial resin) DMF |

DIC/Oxyma, 6 h

| | |
|---|---|
| Coupling | 3.75 eq DIC, 2.50 eq AA, 2.5 eq OxymaPure, |
| | 0.25 mol/L (on AA) in DMF |
| | 2-10 min preactivation |
| | 6 h Coupling time |
| Wash | 4 × 5 min, 6.5 mL/g(initial resin) DMF |

DIC/HOPO, 3 h

| | |
|---|---|
| Coupling | 3.75 eq DIC, 2.50 eq AA, 2.5 eq HOPO, |
| | 0.25 mol/L (on AA) in DMF |
| | 2-10 min preactivation |
| | 3 h Coupling time |
| Wash | 4 × 5 min, 6.5 mL/g(initial resin) DMF |

Final Wash

| | |
|---|---|
| Wash | 6 × 5 min, 6.5 mL/g(initial resin) IPA |
| | 6 × 5 min, 6.5 mL/g(initial resin) MTBE |

TABLE 3
Global Deprotection.

| Step | Protocol Description |
|---|---|
| Resin | The resin bound peptide is placed in the |
| | deprotection vessel weight (kg) = 1 Volume (L) |
| | Cooled Reactor to 0° C. |
| Cocktail | Made a 7 volumes (to weight of resin) cocktail |
| | solution of TFA/water/TIS (95/2.5/2.5, v/v/V) |
| | Pre-cooled cocktail to 0° C. |
| Cleavage | Added cocktail to the peptide-resin and stirred |
| | for 15 min at 5-10° C. |
| | Brought to RT and stirred at RT for 2 h |
| Filtration | Removed spend resin by filtration |
| Resin Wash | Washed spend resin with TFA 2x 0.5 Volumes |
| | (to weight of resin) |
| Precipitation | Brought reaction to 0° C. |
| 1^{st} Addition | Added cold (0° C.) MTBE, 6 volumes (to weight of |
| | resin) slowly (t = 25 min, inner temp < 5° C.) |
| Precipitation | Added cold (0° C.) heptane, 8 volumes (to weight |
| 2^{nd} Addition | of resin) (no control of temperature and addition |
| | speed necessary) |
| | Stirred 15 min at −5 to 0° C. prior to next step |
| Precipitation | Added cold (0° C.) MTBE, 8 volumes (to |
| 3rd Addition | weight of resin) (t = 1 min, T = 0° C.) |
| Ageing | Brought to RT (22-24° C.) |
| | Stirred the solution for 30 min at RT (22-24° C.) |
| Isolation | Filtered the peptide off |
| | Washed 4 times, each with 2 volumes MTBE/heptane (2/1) |
| | Dried under reduced pressure at RT |

TABLE 4
1^{st} pass purification conditions.

| Step | Description |
|---|---|
| Column | YMC Pack C4 HG, 10 μ, 120 Å, 5 × 25 cm |
| Full Load | 8.75 g contained peptide on Ø5 cm |
| Eluents | A: 25 mM NH₄OAc in Water pH 7 (not adjusted, |
| | B: ACN |
| Gradient | 0.0′ - 5% B |
| | 10.0′ - 5% B |
| | 50.0′ - 20% B |
| | 65.0′ - 25% B |
| | 80.0′ - 25% B |
| | 85.0′ - 90% B |
| | 90.0′ - 90% B |
| | 90.1′ - 5% B |
| | 100.0′ - 5% B |
| Flow | 96.5 mL/min |
| Detection | 260 nm |
| Loading | C = 20 mg(crude)/mL dissolved in 500 mM |
| | NH₄HCO₃/ACN (95/5), no pH adjustment |
| Load Volume | V = 1.3-1.0 L (33%-40% content) |
| Fraction | Precharged each fraction vial with 10% Volume |
| Stabilization | of 3M Tris, pH 7 in Water/ACN (95/5), adjusted |
| | with AcOH (Tris free base) |
| Fractions | 1 min Fractions (96.5 mL) Fraction 1-10 |
| | 2 min Fractions (193 mL) Fraction 11-22 |
| | Analyzed with Release method, diluted 1 + 9 |
| | (10%) with Water/ACN (3/1), 1 μL injection, |
| | fractions stored at 5° C. |
| Pooling | Main >94% |
| | Recycling <94%, >50% |
| | Waste <50% |
| Recycling | Fronts and Backs separated, 8.75 g contained |
| | load. |
| | Recycling Pool was diluted with 3xVolume |
| | Eluent A (Fractions at ~20% ACN, dilute |
| | to ~5% ACN) |
| Yield based on | 85% recovery without recycling |
| fraction analysis | |

TABLE 5
2^{nd} pass purification conditions.

| Step | Description |
|---|---|
| Column | YMC Pack C4 HG, 10 μ, 120 Å, 5 × 25 cm |
| Full Load | 8.75 g contained peptide on Ø5 cm |
| Eluents | A: 25 mM NH₄OAc in Water pH 7 |
| | B: ACN |
| Gradient | 0.0′ - 5% B |
| | 5.0′ - 5% B |
| | 20.0′ - 15% B |
| | 40.0′ - 16% B |
| | 70.0′ - 27% B |
| | 70.1′ - 30% B |
| | 80.0′ - 30% B |
| | 80.1′ - 5% B |
| | 90.0′ - 5% B |
| Flow | 96.5 mL/min |
| Detection | 260 nm |
| Loading | 1^{st} Pass Main pool diluted 1 + 3 with 25 mM |
| | NH₄OAc, (pH set to 7 with 3% NH₃, should not |
| | be necessary due to Tris stabilization) |
| Load Volume | 3.5 L after dissolution of fractions |
| Fraction | Precharged each fraction vial with 10% Volume |
| Stabilization | of 3M Tris, pH 7 in Water/ACN (95/5), adjusted |
| | with AcOH (Tris free base) |
| Fractions | 1 min Fractions (96.5 mL), analyzed with Release |
| | method, diluted 1 + 9 (10%) with Water/ACN (3/1), |
| | 1 μL injection, Fractions stored at 5° C. |
| Pooling | Main >96% |
| | Recycling <96%, >50% |
| | Waste <50% |
| Recycling | Fronts and Backs separate, 8.75 g contained load. |
| | Recycling Pool was diluted with 3xVolume |
| | Eluent A (Fractions at ~20% ACN, dilute |
| | to ~5% ACN) |
| Yield based on | 67% recovery without recycling |
| fraction analysis | |

TABLE 6
Concentration pass.

| Step | Description |
|---|---|
| Column | Amberchrome ® CG300 M |
| Full Load | 72 mg contained on a 5 × 30 mm column |
| | Loaded on scale-up column to be updated after |
| | drying of current precipitation |
| Eluents | A: 25 mM NH₄OAc in Water pH 9 |
| | B: ACN |
| Gradient | 0.0′ - 5% B |
| | 20.0′ - 5% B |
| | 20.1′ - 22% B |
| | 60.0′ - 22% B |
| | 60.1′ - 30% B |
| | 80.0′ - 30% B |
| | 80.1′ - 5% B |
| | 100.0′ 5% B |
| Flow | 0.41 mL/min |
| Detection | 260 nm |
| Loading | 2^{st} Pass Main pool diluted 1 + 3 with 25 mM NH₄OAc, |
| | (pH set to 7 with 3% NH₃, should not be necessary |
| | due to Tris stabilization) |
| Load Volume | 1.93 L for scale-up column |
| Fraction | No stabilization - fractions should be stable for at |
| Stabilization | least 24 h at 5° C. (no gelling for scale up for 4 |
| | days) |
| Fractions | 2 Fractions. |
| | 1^{st} fraction: main peak |
| | 2^{nd} fraction: Tail of peak |
| | analyzed with Release method, diluted 1 + 9 = 10% |
| | with Water/ACN = 3/1, 1 μL injection, Fractions |
| | stored at 5° C. |
| Pooling | Main 1^{st} Fraction |
| | Recycling 2^{nd} Fraction |
| Recycling | Not done - but 2^{nd} Fractions could be combined and |
| | re-injected |

TABLE 7
Precipitation, Filtration, and Humidification.

| Precipitation | Description |
|---|---|
| Pool | Fraction from concentration pass - assumption is |
| (Fraction 1) | that ACN content is 22% (water is 78%) |
| | Precipitation performed within 24 h after |
| | concentration pass. |
| Dilution | Made a mixture of AcOH (0.5x Volume of Pool) and |
| Solution | ACN (6.8x Volume of Pool) - Target is 10% water by |
| | addition of ACN |
| Addition | Added dilution solution to Pool under stirring at |
| | RT. Addition speed does not need to be controlled - |
| | addition was relatively quick. |
| MTBE Addition | MTBE (6.8x Volume of Pool) is added under stirring |
| | at RT |
| Ageing | Stirred for 30 min at RT |
| Storage | Precipitation Slurry can be stored at 5° C. under |
| | stirring in order to combine several crude slurries |
| Filtration | |
| Filter Type | 10 μm Nylon Net Filter |
| Vacuum | Applied Δ of 100-300 mbar |
| Drying | Dried at ~5 mbar at 23° C. |
| Homogenization | Dried peptide sample was crunched and sieved |
| Humidification | |
| Wet N₂ stream | Water bath at 19° C., peptide at 23° C. |
| | Humidity of efflux gas reaches ~90% RH but AcOH |
| | is still gassing out of the peptide - humidification |
| | was continued for 48 h |
| Drying | After 48 h humidification at >90% RH the sample was |
| | treated with pure N₂ stream for 48 h. Humidity of |
| | efflux gas dropped to below 10% |

### Comparative Example 1. Comparison of Purity of Materials

The peptide according to SEQ ID NO: 1 of the present invention is synthesized and purified by a conventional method as described below. Tables 8-12 show conditions for the comparative (conventional) method. The comparative SPPS sequence, protocols for SPPS, global deprotection, 1^{st} pass purification, and concentration pass protocols are shown in Tables 8-12, respectively.

TABLE 8
Comparative SPPS Sequence.

| Cycle | AA | Coupling conditions |
|---|---|---|
| 0 | Wang-Resin | Swelling |
| 1 | Fmoc-L-Asp(OtBu)-OH | Loading |
| 2 | Fmoc-L-Ala-L-Ala-OH | Single Coupling - Long |
| 3 | Fmoc-L-Leu-OH | Single Coupling |
| 4 | Fmoc-L-Asp(OtBu)-OH | Single Coupling |
| 5 | Fmoc-Gly-OH | Single Coupling |
| 6 | Fmoc-L-Tyr(tBu)-OH | Single Coupling |
| 7 | Fmoc-L-Ser(tBu)-OH | Single Coupling |
| 8 | Fmoc-L-Leu-OH | Single Coupling |
| 9 | Fmoc-L-Asn(Trt)-L-Thr(psiMe, | Single Coupling - Long |
| | Mepro)-OH | |
| 10 | Fmoc-L-Val-OH | Single Coupling |
| 11 | Fmoc-L-Asn(Trt)-OH | Double Coupling |
| 12 | Fmoc-L-Leu-OH | Double Coupling |
| 13 | Fmoc-Gly-OH | Single Coupling |
| 14 | Fmoc-L-His(Trt)-OH | Double Coupling - His |
| 15 | Fmoc-Gly-OH | Single Coupling |
| 16 | Fmoc-L-Ala * H2O | Single Coupling |
| 17 | Fmoc-L-Pro-OH * H2O | Single Coupling |
| 18 | Fmoc-L-Leu-OH | Single Coupling |
| 19 | Fmoc-L-Glu(OtBu)-OH * H2O | Single Coupling |
| 20 | Fmoc-L-Val-L-Ser(psiMe, | Single Coupling - Long |
| | Mepro)-OH | |
| 21 | Fmoc-L-Lys(Boc)-OH | Single Coupling |
| 22 | Fmoc-L-Asn(Trt)-OH | Double Coupling |
| 23 | Fmoc-L-Val-OH | Double Coupling |
| 24 | Fmoc-L-Val-OH | Double Coupling |
| 25 | Fmoc-L-Val-OH | Double Coupling |
| 26 | Fmoc-L-Ser(tBu)-OH | Double Coupling |
| 27 | Fmoc-Gly-OH | Double Coupling |
| 28 | Boc-L-Asp(OtBu)-OH | Single Coupling, noDeFmoc |
| 29 | — | Final Wash |

TABLE 9
Comparative Protocols for SPPS.
Swelling

| | |
|---|---|
| Resin-Swell | 2 × 10 min, 10 mL/g(inital resin) DMF |

Capping & DeFmoc after Loading

| | |
|---|---|
| Capping | 2.5M Ac₂O in DMF, 2 mL/g(initial resin) |
| | diluted with 8 mL/g(initial resin) DMF |
| | 60 min capping |
| Wash | 1 × 4 min, 10 mL/g(initial resin) DMF |
| | 4 × 4 min, 5 mL/g(initial resin) DMF |
| Deprotection | 20% Piperidine in DMF, 10 mL/g(initial |
| | resin), 20 min |
| | 2 × 4 min, 10 mL/g(initial resin) DMF |
| | 20% Piperidine in DMF, 10 mL/g(initial |
| | resin), 20 min |
| Wash | 1 × 4 min, 10 mL/g(initial resin) DMF |
| | 4 × 4 min, 5 mL/g(initial resin) DMF |

Capping & DeFmoc

| | |
|---|---|
| Capping | 2.5M AC₂O in DMF, 2 mL/g(initial resin) |
| | diluted with 8 mL/g(initial resin) DMF |
| | 10 min capping |
| Wash | 1 × 4 min, 10 mL/g(initial resin) DMF |
| | 4 × 4 min, 5 mL/g(initial resin) DMF |
| Deprotection | 20% Piperidine in DMF, 10 mL/g(initial |
| | resin), 20 min |
| | 2 × 4 min, 10 mL/g(initial resin) DMF |
| | 20% Piperidine in DMF, 10 mL/g(initial |
| | resin), 20 min |
| Wash | 1 × 4 min, 10 mL/g(initial resin) DMF |
| | 4 × 4 min, 5 mL/g(initial resin) DMF |

Coupling
Loading

| | |
|---|---|
| Coupling | 4.0 eq AA, 4.0 eq OxymaPure, 4.0 eq DIC |
| | in DMF, DMAP (cat.) |
| | 12 min preactivation |
| | 4 h Coupling time |
| Wash | 1 × 4 min, 10 mL/g(initial resin) DMF |
| | 4 × 4 min, 5 mL/g(initial resin) DMF |

Single Coupling

| | |
|---|---|
| Coupling | 4.0 eq AA, 4.0 eq OxymaPure, 4.0 eq DIC |
| | 0.25 mol/L (on AA) in DMF |
| | 10 min preactivation |
| | 45 min Coupling time |
| Wash | 1 × 4 min, 10 mL/g(initial resin) DMF |
| | 4 × 4 min, 5 mL/g(initial resin) DMF |

Single Coupling - Long

| | |
|---|---|
| Coupling | 4.0 eq AA, 4.0 eq OxymaPure, 4.0 eq DIC |
| | 0.25 mol/L (on AA) in DMF |
| | 10 min preactivation |
| | 90 min Coupling time |
| Wash | 1 × 4 min, 10 mL/g(initial resin) DMF |
| | 4 × 4 min, 5 mL/g(initial resin) DMF |

Double Coupling

| | |
|---|---|
| 1st Coupling | 4.0 eq AA, 4.0 eq OxymaPure, 4.0 eq DIC |
| | 0.25 mol/L (on AA) in DMF |
| | 10 min preactivation |
| | 45 min Coupling time |
| Wash | 2 × 4 min, 5 mL/g(initial resin) DMF |
| 2nd Coupling | 4.0 eq AA, 4.0 eq OxymaPure, 4.0 eq DIC |
| | 0.25 mol/L (on AA) in DMF |
| | 10 min preactivation |
| | 45 min Coupling time |
| Wash | 1 × 4 min, 10 mL/g(initial resin) DMF |
| | 4 × 4 min, 5 mL/g(initial resin) DMF |

Double Coupling - His

| | |
|---|---|
| 1st Coupling | 4.0 eq AA, 4.0 eq OxymaPure, 4.0 eq DIC |
| | 0.25 mol/L (on AA) in DMF |
| | 2 min preactivation |
| | 45 min Coupling time |
| Wash | 2 × 4 min, 5 mL/g(initial resin) DMF |
| 2nd Coupling | 4.0 eq AA, 4.0 eq OxymaPure, 4.0 eq DIC |
| | 0.25 mol/L (on AA) in DMF |
| | 2 min preactivation |
| | 45 min Coupling time |
| Wash | 1 × 4 min, 10 mL/g(initial resin) DMF |
| | 4 × 4 min, 5 mL/g(initial resin) DMF |

Final Wash

| | |
|---|---|
| Wash | 3 × 4 min, 10 mL/g(initial resin) DCM |
| | 3 × 4 min, 10 mL/g(initial resin) MTBE |

TABLE 10
Comparative Global Deprotection.

| Step | Description |
|---|---|
| Cocktail | Precooled cocktail in ice bath: |
| | TFA/water/TIS/EDT (14.9/0.25/0.25/0.25, each |
| | Volume equivalents) |
| Resin | Added resin to the cocktail while keeping the |
| | temperature below 10° C. weight (kg) = 1 Volume |
| | equivalent (L) |
| Cleavage | Stirred for 2.5 h at 15-25° C. |
| Filtration | Removed spend resin by filtration |
| Resin Wash | Washed spend resin with TFA 3 × 2.5 Volumes |
| | equivalents |
| Precipitation | Brought reaction to <5° C. |
| | Added MTBE, 45.3 Volume equivalents (to weight |
| | of resin) slowly (inner temp <10° C.) |
| Ageing | Brought to 0-10° C. |
| | Stirred the solution for 45 min |
| Isolation | Filtered the peptide off |
| | Washed 3 times, each with 4.8 volume equivalents MTBE |
| Dry | Dried under reduced pressure at RT |
| Trituration | Suspended dry peptide in MTBE (15 Volumes) and |
| | agitate at 0-10° C. for 1.5 h |
| Isolation | Filtered the peptide off |
| | Washed 3 times, each with 5 volume equivalents MTBE |
| Dry | Dried under reduced pressure at RT |

TABLE 11
Comparative 1^{st} pass purification.

| Step | Description |
|---|---|
| Column | Agilent PLRPS 100 Å, 10-15 μ, 8 × 25 cm |
| Eluents | A: ACN |
| | B: 25 mM NH₄OAc in Water/ACN = 95/5 |
| | C: 0.5M NH₄OAc in Water/ACN = 95/5 |
| Gradient | 0.0 CV - 0% A, 0% B, 100% C |
| | 4.9 CV - 0% A, 0% B, 100% C |
| | 0.0 CV - 0% A, 100% B, 0% C |
| | 2.5 CV - 0% A, 100% B, 0% C |
| | 0.5 CV - 10.53% A, 89.47% B, 0% C |
| | 4.0 CV - 11.58% A, 88.42% B, 0% C |
| | 10.5 CV - 15.26% A, 84.74% B, 0% C |
| | 0.5 CV - 36.84% A, 63.16% B, 0% C |
| | 0.5 CV - 94.74% A, 5.26% B, 0% C |
| | 2.0 CV - 94.74% A, 5.26% B, 0% C |
| | 0.2 CV - 0% A, 100% B, 0% C |
| | 3.0 CV - 0% A, 100% B, 0% C |
| Loading | 4.02 g crude peptide in DMSO (9.7 V) and 500 mM |
| | ammonium acetate pH = 8.3 (184 V) - on a 8 |
| | cm diameter column |

TABLE 12
Comparative Concentration Pass.

| Step | Description |
|---|---|
| Column | Agilent PLRPS 100 Å, 10-15 μ, 8 × 25 cm |
| Eluents | A: ACN |
| | B: 10 mM NH₄OAc in Water/ACN = 95/5 |
| Gradient | 0.0 CV - 0% A, 100% B |
| | 2.0 CV - 0% A, 0% B |
| | 1.0 CV - 10.53% A, 89.47% B |
| | 8.0 CV - 26.32% A, 73.68% B |
| | 1.0 CV - 52.63% A, 47.37% B |
| | 0.5 CV - 94.74% A, 5.26% B |
| | 2.0 CV - 94.74% A, 5.26% B |
| | 0.2 CV - 0% A, 100% B |
| | 3.0 CV - 0% A, 100% B |
| Loading | Pooled from first pass, diluted with same volume water |

### Isolation was Performed by Lyophilization.

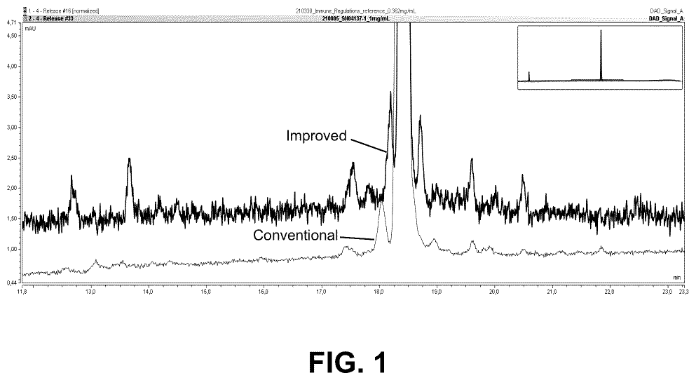

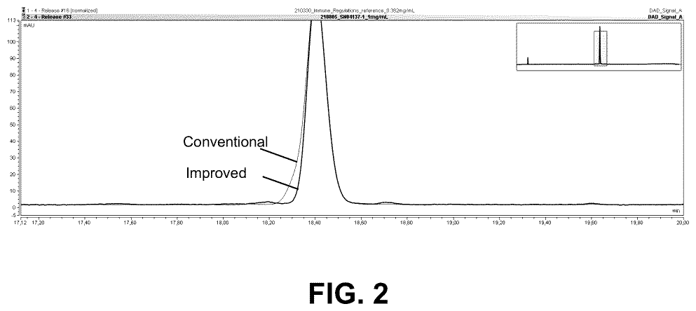

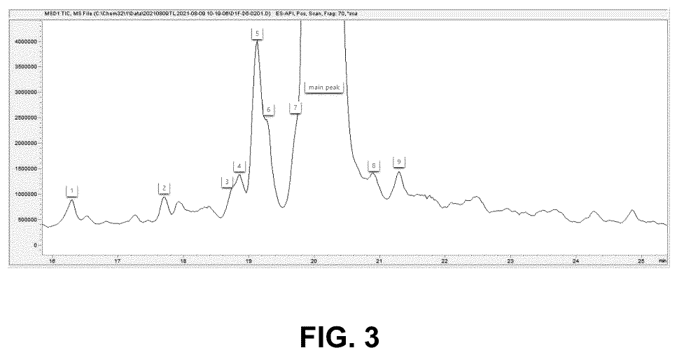

An overlay of chromatograms comparing peptide according to Example 1 and peptide according to Comparative Example 1 is shown in FIG. 1. FIG. 2 is a normalized display of the chromatograms shown in FIG. 1 normalized on the main peak. FIG. 3 is a MS TIC chromatogram of the material produced by a conventional method including the peptide and impurities. Table 13 compares the retention times (minutes) of impurities and product peaks, the area percent purities of each peak, and the AUC (area under curve) for each of the peaks. As used herein the term “impurity” can refer to process and product related impurities including degradation products incurred and is measured (i.e., percent impurity or purity) by area percent as exemplified in Table 13. The purity of the product means the percent area, compared to total integrated peak areas, for the product peak (e.g., Table 13). As such, the purity of the product via the improved method in this example was 96.51%, while the purity of the product via the conventional methods was 89.84%. Notably in this example, the impurity peak at 17.918 minutes (5.53 area %) in the conventional method was not present using the improved method. This front peak was difficult to remove by preparative chromatography.

TABLE 13
Comparative Purity of Product.

| Improved | Peak | Ret. Time | Area | AUC (area under |
|---|---|---|---|---|
| Method: | #/Desc.: | (min.): | %: | curve) mAU*min: |
| | 1 (impurity) | 12.66 | 0.27 | 0.0361 |
| | 2 (impurity) | 13.668 | 0.54 | 0.0719 |
| | 3 (impurity) | 17.536 | 0.50 | 0.0656 |
| | 4 (impurity) | 18.195 | 1.00 | 0.1324 |
| | 5 (product) | 18.403 | 96.51 | 12.7519 |
| | 6 (impurity) | 18.708 | 0.74 | 0.0978 |
| | 7 (impurity) | 19.61 | 0.28 | 0.0364 |
| | 8 (impurity) | 20.48 | 0.16 | 0.0214 |

| Conventional | Peak | Ret. Time | Area | AUC (area under |
|---|---|---|---|---|
| Method: | #/Desc.: | (min.): | %: | curve) mAU*min: |
| | 1 (impurity) | 3.688 | 0.9 | 0.0898 |
| | 2 (impurity) | 12.682 | 0.13 | 0.0128 |
| | 3 (impurity) | 13.148 | 0.09 | 0.0087 |
| | 4 (impurity) | 15.56 | 0.01 | 0.001 |
| | 5 (impurity) | 17.047 | 0.28 | 0.0282 |
| | 6 (impurity) | 17.642 | 1.21 | 0.1205 |
| | 7 (impurity) | 17.918 | 5.53 | 0.5516 |
| | 8 (product) | 18.003 | 89.84 | 8.9613 |
| | 9 (impurity) | 18.145 | 1.03 | 0.1023 |
| | 10 (impurity) | 18.542 | 0.46 | 0.0458 |
| | 11 (impurity) | 19.223 | 0.22 | 0.0218 |
| | 12 (impurity) | 19.505 | 0.18 | 0.0182 |
| | 13 (impurity) | 20.103 | 0.06 | 0.0057 |
| | 14 (impurity) | 21.443 | 0.04 | 0.0044 |
| | 15 (impurity) | 23.685 | 0.03 | 0.0026 |

Table 14 shown below provides further details on the MS peaks seen in the chromatogram of the material produced by a conventional method (FIG. 3).

TABLE 14
Comparative MS Peaks.

| Peak | Mass | Comments |
|---|---|---|
| main peak | M + 0 | Peptide of interest |
| 1 | M + 127 | +Lys |
| 2 | M − 415 | unknown |
| 3 | M − 57 | −Gly |
| 4 | Main peaks: M + 137, M + 57 | +His, +Gly |
| 5 | M + 57, M + 0 | +Gly, isomer |
| 6 | M + 87 | +Ser |
| 7 | M + 0 | isomer |
| 8 | M + 0 | isomer |
| 9 | M + 0, M + 71 | isomer, +Ala |

As demonstrated by the Example 1 and Comparative Example 1, the material produced by Example 1 advantageously increases purity of the peptide of interest, i.e., includes less impurities. The method of Example 1 also has multiple advantages over the conventional method of Comparative Example 1, including using less raw materials, less volume of solvent, less volume of the cleavage cocktail, less volume of the antisolvent, eliminating the step of trituration, increasing loading during purification to enhance processability, and adding a concentration step to improve precipitation.

### Comparison Summary

The improved method provided surprisingly high purity and purification (column) loading because front-eluting peaks, difficult to purify out, were not present. The formula weight of SEQ ID NO. 1 was estimated to be 3112.41 Da, and the exact mass at 3110.55 Da (molecular formula C₁₃₄H₂₁₅N₃₇O₄₈). The protected FW was estimated at 4754.81 Da (molecular formula C₂₅₄H₃₅₁N₃₇O₅₂). Example differences and advantages of the methods of the present invention over the conventional methods are summarized in Tables 15-18 below.

TABLE 15
Example SPPS Comparison.
SPPS

| | | |
|---|---|---|
| | | Difference/ |
| Present Disclosure | Conventional | Justification |

Coupling (both DIC/Oxyma activation in DMF)

| | | |
|---|---|---|
| 4 eq AA | 2.5 ea AA | 144 eq vs 70 eq total |
| 18 single couplings (45 min) | 28 single couplings | −50% raw materials |
| 9 Double couplings (2 × 45 min) | (12 × 3 h + 16 × 6 h) | (Fmoc-AAs, Coupling |
| | | agents) |

DeFmoc

| | | |
|---|---|---|
| 20% Piperidin in DMF | 10% Piperidin in DMF + | Similar Volume but |
| 2x 10 Volumes (2 × 20 min) | 0.15M Oxyma | 50% less piperidin |
| | 3 × 6.5 Volumes (3 × 10 min) | |

Capping

| | | |
|---|---|---|
| Yes (1 × 10 min) | No | No reagents for |
| | | capping needed (raw |
| | | materials/cleaning |
| | | of equipment not |
| | | necessary) |

Wash per Cycle

| | | |
|---|---|---|
| 20 × 5 Volumes (17 × 4 min) | 9 × 6.5 Volumes (9 × 5 min) | 2.700 Volumes vs |
| 100 volumes/cycle | 59 volumes/cycle | 1652 Volumes |
| | | −39% Volumes DMF |

Total time

| | | |
|---|---|---|
| 81 h - without transfers | 140 h - without transfers | +1.7x - without |
| Cycle time~5 h (x18) | Cycle time~7 h (x12) & | transfers |
| 6 h (x9) | 10 h (x16) | Total assumption 144 |
| | | h vs 244 h + 1.7x |

TABLE 16
Example Global Deprotection.
Global Deprotection

| | | |
|---|---|---|
| | | Difference/ |
| Present Disclosure | Conventional | Justification |

Cleavage

| | | |
|---|---|---|
| TFA/TIS/Water/EDT - 15.6 | TFA/TIS/Water - 7 Volumes | Cleavage Volume |
| Volumes | 2.5 h | reduction: 65% less |
| 2.5 h | TFA wash 2 × 0.5 Volumes | EDT not used |
| TFA wash 3 × 2.5 Volumes | Total Volume: 8 Volumes | (problematic due to |
| Total Volume: 23 Volumes | | odor, no scientific |
| | | rationale why EDT is |
| | | necessary for |
| | | scavenging) |

Precipitation

| | | |
|---|---|---|
| 1. MTBE - 45.3 Volumes | 1. MTBE - 6 Volumes | Anti-Solvent Volume |
| Wash: 3 × 4.8 Volumes MTBE | 2. Heptan - 8 Volumes | reduction: 50% less |
| Total Volume: 59 Volumes | 3. MTBE - 8 Volumes | Filterability is |
| | Wash: 4 × 2 Volumes MTBE/Heptan | enhanced by step- |
| | Total Volume: 30 Volumes | wise addition |
| | | (combining of anti- |
| | | solvents causes |
| | | gelling of particles |
| | | and slows down |
| | | filtration) |

Drying

| | | |
|---|---|---|
| Similar | Similar | — |

Trituration and Drying

| | | |
|---|---|---|
| MTBE: 15 Volumes - 1.5 h | No | Not needed - no re- |
| Wash: 3 × 5 Volumes | | Drying needed |

Total Volume

| | | |
|---|---|---|
| 112 Volumes | 38 Volumes | Only 34% of RM |
| | | needed |

TABLE 17
Example Purification Steps 1^{st} and 2^{nd} Pass.
Purification steps 1^{st} and 2^{nd} pass

| | | |
|---|---|---|
| | | Difference/ |
| Present Disclosure | Conventional | Justification |

Column Material

| | | |
|---|---|---|
| Agilent PLRPS 100 A 10-15 μ | YMC Pack C4 HG, 10 μ, 120 A | Potentially higher |
| | | load to be achieved |

Mobile Phase

| | | |
|---|---|---|
| A: 25 mM & 10 mM NH4OAc | A: 25 mM NH4OAc pH 7 | No major diference |
| B: ACN | B: ACN | |

Loading

| | | |
|---|---|---|
| Up to 4 g crude on 8 cm | Up to 22.4 g contained on 8 cm | Higher loading |
| (~1.6 g contained) | (~40 g crude) | enhances |
| | | processability |

Stabilization of 1st & 2nd pass fractions

| | | |
|---|---|---|
| None (gelled fractions were | 3M Tris pH 7 (10 V % added to | No gelling of fractions |
| re-dissolved by pH adjust after | Fractions) | is mandatory for CPC |
| visual inspection) | | production (no visual |
| | | control possible) |
| | | Prevention of gelling |
| | | might be less invasive |
| | | compared to break- |
| | | up of gel |

TABLE 18
Example Concentration and Isolation.
Concentration and Isolation

| | | |
|---|---|---|
| | | Difference/ |
| Present Disclosure | Conventional | Justification |

Concentration

| | | |
|---|---|---|
| Not done | Concentrated on Amberchrome CG300M | Higher concentration |
| | with NH₄OAc/ACN pH 9 | with low water |
| | | content for |
| | | precipitation |

Isolation

| | |
|---|---|
| Lyophilization | Precipitation |
| | 1. Dilute with 0.5x Volume AcOH |
| | premixed with ACN (ACN Volume to |
| | target 10% final water content) |
| | 2. Dilute with MTBE (same Volume as |
| | for ACN) |
| | 3. Age for 30 min or hold at 5° C. |
| | 4. Filter off precipitate over 10 μ Nylon |
| | membrane filter |
| | 5. Wash with MTBE |

Humidification

| | | |
|---|---|---|
| Not needed | 1. Humidify with wet N₂ until ~90% RH | Residual solvents |
| | is reached | (AcOH, MTBE & ACN) |
| | 2. Dry with N₂ stream | from precipitation |
| | | are removed |

### Definitions

The terms and abbreviations used in the instant specification will have the meaning as provided herein. If a particular term is not defined herein, it will have the meaning as generally known to one skilled in the art.

The term “DMF” represents the solvent dimethyl formamide.

The term “elute” refers to eluant or eluate.

The term “Oxyma” as used herein represents ethyl 2-cyano-2-(hydroxyamino)acetate.

The term Fmoc” as used herein represents the protecting group 9-fluorenylmethyloxycarbonyl group, and the term “Boc” or “t-Boc” is intended to represent a t-butyloxycarbonyl group. The Boc and Fmoc groups are used to protect the amino group/terminus of an amino acid during the solid phase peptide synthesis (SPPS).

The term “SPPS” represents solid phase peptide synthesis, a synthetic method used to synthesize peptides.

The term “AA” as used herein represents any amino acid.

The letter “D” represents Aspartic Acid.

The letter “A” represents Alanine.

The letter “G” represents Glycine.

The letter “S” represents Serine.

The letter “V” represents Valine.

The letter “N” represents Asparagine.

The letter “K” represents Lysine.

The letter “E” represents Glutamine.

The letter “H” represents Histidine.

The letter “L” represents Leucine.

The letter “T” represents Threonine.

The letter “Y” represents Tyrosine.

The term “TIS” represents triisopropylsilane.

The Term “TFA” represents tri-fluoro acetic acid.

The embodiments of the disclosure described above are intended to be merely exemplary; numerous variations and modifications will be apparent to those skilled in the art. All such variations and modifications are intended to be within the scope of the present disclosure as defined in any appended claims.

All references referred to in the present disclosure are hereby incorporated by reference in their entirety. Various embodiments of the present disclosure may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

### REFERENCES

¹ Ranford, J. C., et al. (2000). “Chaperonins are cell signalling polypeptides: —the unfolding biology of molecular chaperones.” Exp. Rev. Mol. Med., 15; 2(8):1-17.
² Ranson, N., et al. (1998). “Review Article: Chaperones.” Biochem. J. 333: 233-242.
³ Amblard, M., et al. (2006). “Methods and protocols of modern solid phase peptide synthesis.” Molecular Biotechnology, 33(3): 239-254.
⁴ Behrendt, R., et al. (2016). “Advances in Fmoc solid-phase peptide synthesis.” Journal of Peptide Science, 22(1): 4-27, 1075-2617

What is claimed is:

1. A process of synthesizing a peptide with SEQ ID NO:1 (DGSVVVNKVSELPAGHGLNVNTLSYGDLAAD), the process comprising the steps of:
(i) attaching an amino acid (AA) to a resin of a solid support via the AA's C terminus and wherein the N terminus of the amino acid is protected with a protecting group to avoid reaction at the N terminus to form a first solid support bound AA;
(ii) deprotecting the N terminus of the first solid support bound AA by removing the protecting group;
(iii) coupling a second AA with the first solid support bound AA wherein the C terminus of the second amino acid is coupled with the de-protected N terminus of the first solid support bound AA to form a second solid support bound AA, and wherein the second AA comprises a protected N terminus;
(iv) repeating steps (ii) and (iii) to form the next solid support bound AA until a solid support bound AA sequence comprising SEQ ID NO: 1 is formed;
(v) cleaving the AA sequence from the solid support to yield a peptide mixture of the peptide comprising SEQ ID NO: 1;
(vi) separating the peptide mixture from the solid support by filtration to yield a crude product;
(vii) diluting the separated peptide mixture with solvents to form a precipitate, wherein the precipitate is isolated by filtration;
(viii) subjecting the isolated precipitate to column chromatography to collect eluant fractions comprising a purified version of the peptide;
(ix) concentrating the eluant fractions to form a concentrated eluate comprising the purified version of the peptide; and
(x) isolating the peptide from the concentrated eluate by precipitation followed by filtration.

2. The process of claim 1 wherein the steps (vii) and/or (x) include precipitating the crude product and/or the eluate via a stepwise adding of methyl tert-butyl ether (MTBE) and heptane; and wherein the stepwise adding enhances filterability by preventing gelling, whereas executing (vii) and/or (x) without the stepwise adding by combining the antisolvents causes gelling of precipitate, stops the filtration, and prevents any scale-up of the method.

3. The process of claim 2, wherein in step (i), the C terminus of the AA Alanine (A) is attached to a resin bound amine of aspartic acid from the solid support by treating about 2.5 equivalent of the AA (A) with 2-(1H-benzotriazole-1-yl)-1,1,3,3-tetramethyluronium tetrafluoroborate (TBTU) and N,N-diisopropylethylamine (DIEA) in DMF, and further wherein the N-terminus of the AA (A) is protected by using Fmoc as the protecting group.

4. The process of claim 3, wherein in step (ii), an Fmoc protected N-terminus of a dipeptide (AD) is deprotected by three consecutive treatments of the solid support bound dipeptide (AD) with a mixture of 10% piperidine in DMF and 0.15 M Oxyma.

5. The process of claim 4, wherein in step (ii), an Fmoc protected N terminus of a dipeptide (AD) is deprotected by three consecutive treatments of the solid support bound dipeptide (AD) with a mixture of 10% piperidine in DMF and 0.15 M Oxyma, and wherein the solid support bound dipeptide (AD) comprising a deprotected N-terminus is treated with about 2.5 equivalent of a second AA—Alanine (A) in the presence of N,N-diisopropylcarbodiamide (DIC) and ethyl 2-cyano-2-(hydroxyamino)acetate (Oxyma) in DMF, and further wherein the N-terminus of the second AA (A) is protected by using Fmoc as the protecting group.

6. The process of claim 5, wherein the N-terminus of the second AA (A) is deprotected by three consecutive treatments of the solid support bound AA's with a mixture of 10% piperidine in DMF and 0.15 M Oxyma.

7. The process of claim 6, wherein the solid support bound AA's with the deprotected N-terminus is sequentially treated with the steps in (ii) and (iii) until a solid supported peptide is formed with the SEQ ID NO:1.

8. The process of claim 7, wherein the peptide with SEQ ID NO: 1 is obtained by cleaving the solid supported peptide with SEQ ID NO: 1 from the solid support by treating the solid supported peptide with SEQ ID NO: 1 with an aqueous solution comprising trifluoroacetic acid (TFA) and triisopropylsilane (TIS), and separating the peptide with SEQ ID NO: 1 from the solid support by passing the mixture through a filter wherein the peptide with SEQ ID NO: 1 passes through the filter in to the filtrate, and wherein the filtered solid material is further washed up to eight times with the aqueous solution comprising TFA and TIS to yield a filtrate comprising the peptide with SEQ ID NO: 1.

9. The process of claim 8, wherein in step (x), the elute is diluted with stepwise addition of methyl tert-butyl ether (MTBE), heptane, and MTBE, in a ratio of 1:0.75:1:1 by volume to yield the peptide with SEQ ID NO:1 as a precipitate.

10. The process of claim 9, wherein the SEQ ID NO:1 peptide precipitate is further subjected to a reverse phase high performance liquid column chromatography using a C₄ reversed phase column.

11. The process of claim 10, wherein the SEQ ID NO: 1 peptide precipitate is further subjected to a reverse phase high performance liquid column chromatography using a C₄ reversed phase column, and wherein a pore size of the C₄ reversed phase column ranges from 100-120 Å.

12. The process of claim 11, wherein the SEQ ID NO: 1 peptide precipitate is further subjected to a reverse phase high performance liquid column chromatography using a C₄ reversed phase column, and wherein a particle size of the C₄ reversed phase column is 10 μm.

13. The process of claim 12, wherein the SEQ ID NO: 1 peptide precipitate is further subjected to a reverse phase high performance liquid column chromatography using a C₄ reversed phase column, and wherein a mobile phase A is 25 mM to 50 mM ammonium acetate at a pH of 7 to 8.4; and wherein a mobile phase B is acetonitrile (ACN).

14. The process of claim 13, wherein the SEQ ID NO: 1 peptide precipitate is further subjected to a reverse phase high performance liquid column chromatography using a C₄ reversed phase column, and wherein a mobile phase B is acetonitrile (ACN).

15. The process of claim 14, wherein the SEQ ID NO: 1 peptide precipitate is further subjected to a reverse phase high performance liquid column chromatography using a C₄ reversed phase column, and wherein purifying the peptide precipitate by a reverse phase high performance liquid column chromatography using a C₄ reversed phase column further comprises loading the C₄ reversed phase column to a concentration of 23 mg of crude product per mL of stationary phase.

16. The process of claim 1, wherein the SEQ ID NO: 1 peptide precipitate is further subjected to a reverse phase high performance liquid column chromatography using a C₄ reversed phase column; and wherein the C₄ reverse phase column bed has a height of from 20 cm to 40 cm.

17. The process of claim 16, wherein the SEQ ID NO: 1 peptide precipitate is further subjected to a reverse phase high performance liquid column chromatography using a C₄ reversed phase column, and wherein the mobile phases are collected as eluants after passing through the C₄ reverse phase column, and further wherein the eluants are diluted with 10% of tris(hydroxymethyl)aminomethane (Tris) in water at a pH of 7 to yield the peptide.

18. The process of claim 1, wherein isolating the purified product from the concentrated eluate includes diluting the concentrated eluate with 0.5× volume acetic acid (AcOH) premixed with ACN to form a reaction mixture.

19. The process of claim 17, wherein the peptide is diluted using MTBE to form a mixture, and wherein isolating the peptide from the mixture further comprises aging the reaction mixture for 30 minutes at 5° C. to yield a heterogenous mixture.

20. The process of claim 19, further comprising the process is a process of synthesizing a peptide with a desired sequence ID; and wherein (iv) and (v) are further comprising and are replaced as shown below:
(iv) repeating steps (ii) and (iii) to form the next solid support bound AA until a solid support bound AA sequence including the desired sequence ID formed; and
(v) cleaving the AA sequence from the solid support to yield a peptide mixture of the peptide comprising the desired sequence ID.

21. The process of claim 20, wherein the peptide from the heterogenous mixture is isolated by filtering the heterogenous mixture through a nylon membrane filter.

22. The process of claim 21, wherein the nylon membrane filter is a 10 μm nylon membrane.

23. The process of claim 22, wherein the isolated peptide is further washed with MTBE.

24. The process of claim 23, further comprising humidifying the peptide to remove residual solvents.

25. The process of claim 24, wherein humidifying the peptide molecule includes humidifying the peptide molecule with wet N₂ until about 90% relative humidity is reached, followed by drying with a N₂ stream to yield the peptide in a dry form.
